(19) United States
(12) Patent Application Publication
(10) Pub. No.: US 2018/0315505 A1
(43) Pub. Date: Nov. 1, 2018

(54) OPTIMIZATION OF CLINICAL DECISION MAKING

(71) Applicant: Siemens Healthcare GmbH, Erlangen (DE)

(72) Inventors: Lucian Mihai Itu, Brasov (RO); Puneet Sharma, Monmouth Junction, NJ (US); Razvan Ionasec, Nuernberg (DE); Dorin Comaniciu, Princeton Junction, NJ (US)

(21) Appl. No.: 15/944,676

(22) Filed: Apr. 3, 2018

Related U.S. Application Data
(60) Provisional application No. 62/490,629, filed on Apr. 27, 2017.

(51) Int. Cl. G16H 50/20; G16H 10/20; G16H 10/60

(52) U.S. Cl. CPC G16H 50/20; G16H 10/60; G16H 10/20

(57) ABSTRACT

Systems and methods for optimizing the decision to perform additional clinical testing are provided. A model of cutoff values, associated with the initial clinical test and representing a tradeoff between a plurality of factors, is generated. Each of the cutoff values define a boundary within a range of results of the initial clinical test delineating results that provide a medical evaluation and results that do not provide the medical evaluation. At least one optimized cutoff value associated with the initial clinical test is determined from the cutoff values by optimizing the model based on the tradeoff between the plurality of factors. It is determined whether to perform the additional clinical test based on a result of the initial clinical test performed on the patient and the at least one optimized cutoff value.

### CROSS-REFERENCE TO RELATED APPLICATIONS

This application claims the benefit of U.S. Provisional Application No. 62/490,629, filed Apr. 27, 2017, the disclosure of which is herein incorporated by reference in its entirety.

### BACKGROUND

The present invention relates generally to the optimization of clinical decision making, and more particularly to optimizing the decision to perform additional clinical tests on a patient based on a result of an initial clinical test relative to optimized cutoff points.

Traditionally, coronary artery disease (CAD) has been evaluated based on coronary computed tomography angiography (CTA) imaging of a patient. CTA imaging includes a significant amount of information of the patient, such as, e.g., calcium, plaque, degree and morphology of stenosis, etc. Utilizing this information for evaluating CAD of the patient may help reduce the need for more time consuming, costly, or invasive additional testing. However, the information in the CTA imaging is not completely utilized during the traditional clinical evaluation of the patient for CAD.

Furthermore, CAD is evaluated based on the relatively low specificity of CTA imaging according to the subjective analysis of the clinician. Accordingly, a patient with intermediate lesions identified from CTA imaging may be further evaluated with additional, more advanced testing. Typically, the decision of whether to undergo additional testing is determined based on fixed cutoff points associated with the outcome of the initial testing so as to maximize certain statistical measures, such as, e.g., accuracy. For example, if a stenosis grading in CTA imaging is between the fixed cutoff points of 30% and 90%, computed tomography-fractional flow reserve (CT-FFR) may be employed. CT-FFR is a costly and time consuming process. The fixed cutoff points for determining whether to employ the additional testing of CT-FFR do not consider the treatment cost or the patient outcome in determining whether to employ additional testing.

### BRIEF SUMMARY OF THE INVENTION

In accordance with one or more embodiments, systems and methods for optimizing the decision to perform additional clinical testing are provided.

In accordance with one or more embodiments, a method comprises: generating a model of cutoff values associated with an initial clinical test and representing a tradeoff between a plurality of factors, each of the cutoff values defining a boundary within a range of results of the initial clinical test delineating results that provide a medical evaluation and results that do not provide the medical evaluation; determining at least one optimized cutoff value associated with the initial clinical test from the cutoff values by optimizing the model based on the tradeoff between the plurality of factors; and determining whether to perform an additional clinical test based on a result of the initial clinical test performed on the patient and the at least one optimized cutoff value.

In accordance with one or more embodiments, the plurality of factors comprises accuracy of the initial clinical test, cost of the initial clinical test, and patient outcome of the initial clinical test.

In accordance with one or more embodiments, determining whether to perform an additional clinical test based on a result of the initial clinical test performed on the patient and the at least one optimized cutoff value comprises: a) performing the initial clinical test on a patient; b) determining whether a result of the initial clinical test performed on the patient provides a medical evaluation of the patient based on the at least one optimized cutoff value; and c) in response to determining that the result of the initial clinical test performed on the patient does not provide the medical evaluation of the patient, repeating steps a) and b) using a respective additional clinical test as the initial clinical test until it is determined that the result of the respective clinical test performed on the patient provides the medical evaluation of the patient or for a predetermined number of iterations.

In accordance with one or more embodiments, the at least one optimized cutoff value associated with the initial clinical test comprises: an optimized lower cutoff value delineating results below the optimized lower cutoff value that provide the medical evaluation and results above the optimized lower cutoff value that do not provide the medical evaluation; and an optimized upper cutoff value delineating results above the optimized upper cutoff value that provide the medical evaluation and results below the optimized upper cutoff value that do not provide the medical evaluation.

In accordance with one or more embodiments, determining whether a result of the initial clinical test performed on the patient provides a medical evaluation of the patient based on the at least one optimized cutoff value comprises: determining that the result of the initial clinical test performed on the patient does not provide the medical evaluation of the patient when the result of the initial clinical test is above the optimized lower cutoff value and below the optimized upper cutoff value.

In accordance with one or more embodiments, the additional clinical test is more expensive to perform on the patient than the initial clinical test.

In accordance with one or more embodiments, the initial clinical test comprises at least one trained machine learning model.

In accordance with one or more embodiments, the at least one trained machine learning model is trained to predict the result of the initial clinical test or whether to apply the additional clinical test.

In accordance with one or more embodiments, the at least one trained machine learning model comprises a cascade of trained machine learning models, wherein the trained machine learning models in the cascade are successively applied until it is determined whether to apply the additional clinical test.

In accordance with one or more embodiments, an apparatus comprises: means for generating a model of cutoff values associated with an initial clinical test and representing a tradeoff between a plurality of factors, each of the cutoff values defining a boundary within a range of results of the initial clinical test delineating results that provide a medical evaluation and results that do not provide the medical evaluation; means for determining at least one optimized cutoff value associated with the initial clinical test from the cutoff values by optimizing the model based on the tradeoff between the plurality of factors; and means for determining whether to perform an additional clinical test based on a result of the initial clinical test performed on the patient and the at least one optimized cutoff value.

In accordance with one or more embodiments, a non-transitory computer readable medium stores computer program instructions, the computer program instructions when executed by a processor cause the processor to perform operations comprising: generating a model of cutoff values associated with an initial clinical test and representing a tradeoff between a plurality of factors, each of the cutoff values defining a boundary within a range of results of the initial clinical test delineating results that provide a medical evaluation and results that do not provide the medical evaluation; determining at least one optimized cutoff value associated with the initial clinical test from the cutoff values by optimizing the model based on the tradeoff between the plurality of factors; and determining whether to perform an additional clinical test based on a result of the initial clinical test performed on the patient and the at least one optimized cutoff value.

These and other advantages of the invention will be apparent to those of ordinary skill in the art by reference to the following detailed description and the accompanying drawings.

### BRIEF DESCRIPTION OF THE DRAWINGS

FIG. 1 shows an exemplary system for optimizing clinical decision making for performing one or more additional clinical tests, in accordance with one or more embodiments;

FIG. 2 shows a workflow for performing a medical evaluation on a patient, in accordance with one or more embodiments;

FIG. 3 shows a method for determining optimized cutoff values for an initial clinical test, in accordance with one or more embodiments;

FIG. 4 shows an exemplary three dimensional surface model, in accordance with one or more embodiments;

FIG. 5 shows a method for performing a medical evaluation on a patient, in accordance with one or more embodiments;

FIG. 6 shows a workflow for training and applying a machine learning model for predicting a measure of interest, in accordance with one or more embodiments;

FIG. 7 shows a user interface, in accordance with one or more embodiments;

FIG. 8 shows a workflow for hierarchically applying a cascade of trained machine learning models, in accordance with one or more embodiments; and

FIG. 9 shows a high-level block diagram of a computer, in accordance with one or more embodiments.

### DETAILED DESCRIPTION

The present invention generally relates to optimizing the decision to perform additional clinical tests on a patient based on a result of an initial clinical test relative to optimized cutoff points. Embodiments of the present invention are described herein to give a visual understanding of methods for optimizing contrast imaging of a patient. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

Further, it should be understood that while the embodiments discussed herein may be discussed with respect to the clinical decision making process for a medical evaluating a patient, the present invention is not so limited. Embodiments of the present invention may be applied for any type of decision making process for any subject.

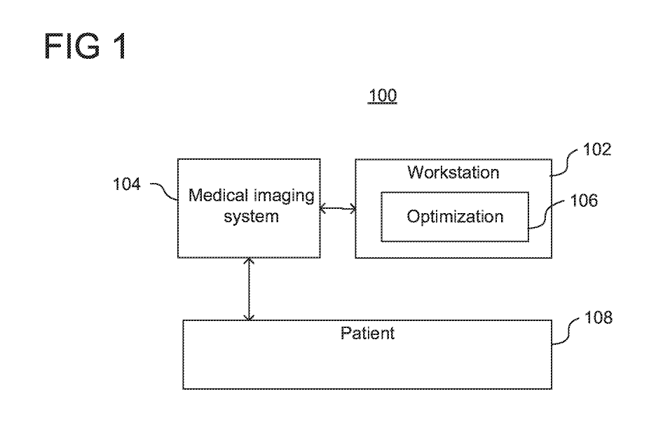

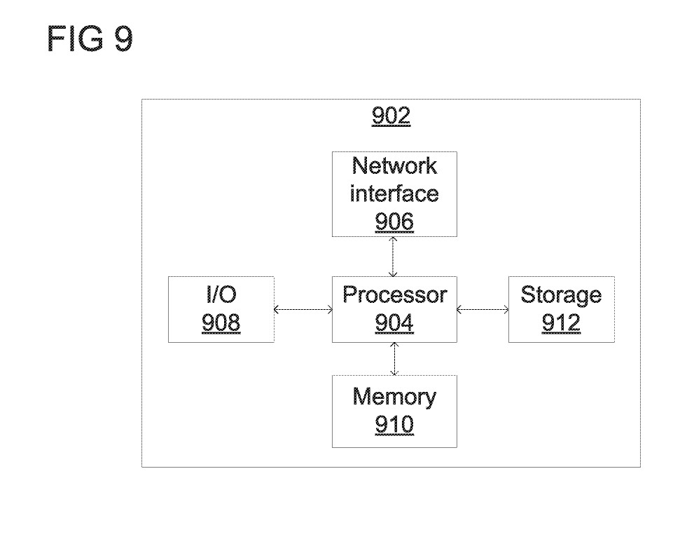

FIG. 1 shows a system 100 configured for optimizing clinical decision making for performing one or more additional clinical tests to perform a medical evaluation on a patient, in accordance with one or more embodiments. System 100 includes workstation 102, which may be used for assisting a clinician (e.g., a doctor, a medical professional, or any other user) for performing a medical evaluation on a patient 114. Workstation 102 may be implemented using any suitable computing device, such as, e.g., computer 902 of FIG. 9.

Workstation 102 may assist the clinician in performing a medical evaluation of patient 108 by performing one or more clinical tests. For example, workstation 102 may receive imaging data of patient 108 from medical imaging system 104 for performing the clinical test. Medical imaging system 104 may be of any modality, such as, e.g., x-ray, magnetic resonance imaging (MRI), computed tomography (CT), ultrasound (US), single-photon emission computed tomography (SPECT), positron emission tomography (PET), or any other suitable modality or combination of modalities. In another embodiment, workstation 102 may receive the imaging data by loading previously stored imaging data of the patient acquired using medical imaging system 104.

In one embodiment, workstation 102 assists the clinician in performing a medical evaluation of patient 108 for diagnosing patient 108 with CAD (coronary artery disease) by performing one or more clinical tests. In one example, the one or more clinical tests include CTA (computed tomography angiograph). Accordingly, CT-based medical imaging system 104 scans patient 108. A contrast agent (e.g., iodine, barium, gadolinium, microbubble, etc.) may be injected into patient 108 to enhance the visibility of blood vessels or organs of patient 108 in the CTA imaging. Anatomical features in the CTA imaging may be analyzed to determine a result of the CTA. For example, a result of the CTA may be a stenosis grade having a range of results from 0% to 100% indicating no restriction of blood flow in the artery to complete restriction of blood flow in the artery, respectively. A result of the CTA performed on the patient below a lower cutoff value in the range of results can rule out CAD while a result above an upper cutoff value in the range of results can rule in CAD. However, a result having a value between the lower and upper cutoff values in the range of results cannot provide the medical evaluation of CAD with an appreciate level of certainty and may be a candidate for one or more additional clinical tests.

Conventionally, the values of the lower and upper cutoff values are predetermined, fixed values, typically determined to maximize a certain statistical measure (e.g., accuracy). For example, the lower and upper cutoff values for evaluating CTA for CAD may be 30% and 90%, respectively. However, such conventional approaches for determining cutoff values do not consider other factors, such as, e.g., cost and patient outcome.

Advantageously, optimization module 106, implemented on workstation 102 of FIG. 1, optimizes the decision to perform additional clinical tests on a patient based on a result of an initial clinical test relative to optimized cutoff points. In particular, optimization module 106 is configured to determine optimized cutoff values associated with an initial clinical test based on a number of different factors, such as, e.g., statistical measures (e.g., accuracy, sensitivity, specificity), patient outcome, and costs. Accordingly, the optimized cutoff values represent a tradeoff between the, e.g., statistical measures, patient outcome, and costs. Clinical decision support is provided by the optimized cutoff values by determining whether to perform an additional test on the patient based on the result of the initial clinical test with respect to the optimized cutoff values.

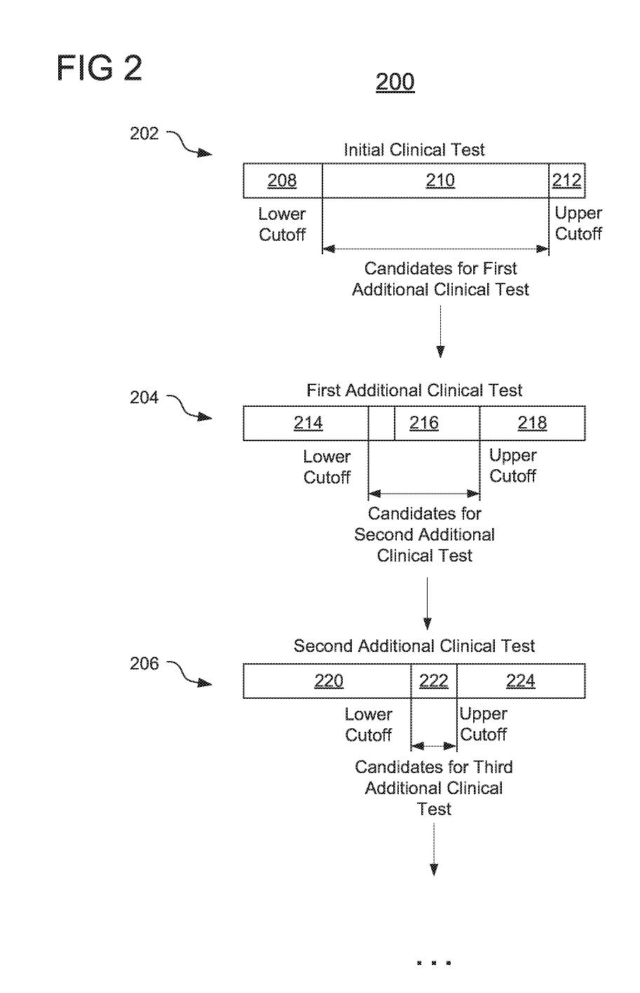

Referring to FIG. 2, a workflow 200 for performing a medical evaluation on a patient by applying one or more additional clinical tests is shown, in accordance with one or more embodiments. In one example, the medical evaluation is the diagnosis of the patient with CAD. An initial clinical test is first performed on a patient. The initial clinical test may be, e.g., CTA, having a range of results 202. In one embodiment, range of results 202 of CTA corresponds to a stenosis grading ranging from 0% to 100% or ranging from grade 0 to grade 4. The result of the clinical test may be in any suitable format.

Range of results 202 includes a lower cutoff value and an upper cutoff value. The cutoff values define a boundary within range of results 202 of the clinical test delineating results that provide a medical evaluation and results that do not provide the medical evaluation. For example, range of results 202 includes a lower cutoff value and an upper cutoff value. The lower cutoff value delineates results 208 (below the lower cutoff value) that provide a medical evaluation ruling out CAD from results 210 (above the lower cutoff value) that do not provide the medical evaluation ruling out (or ruling in) CAD. Similarly, the upper cutoff value delineates results 212 (above the upper cutoff value) that provide a medical evaluation ruling in CAD and results 210 (below the upper cutoff value) that do not provide the medical evaluation ruling in (or ruling out) CAD. A result of the CTA performed on the patient within results 210 (between the lower and upper cutoff values) would not provide the medical evaluation ruling in or ruling out CAD and would therefore be a candidate for one or more additional clinical tests. For example, a patient having a result of the CTA within results 210 would undergo a first additional clinical test.

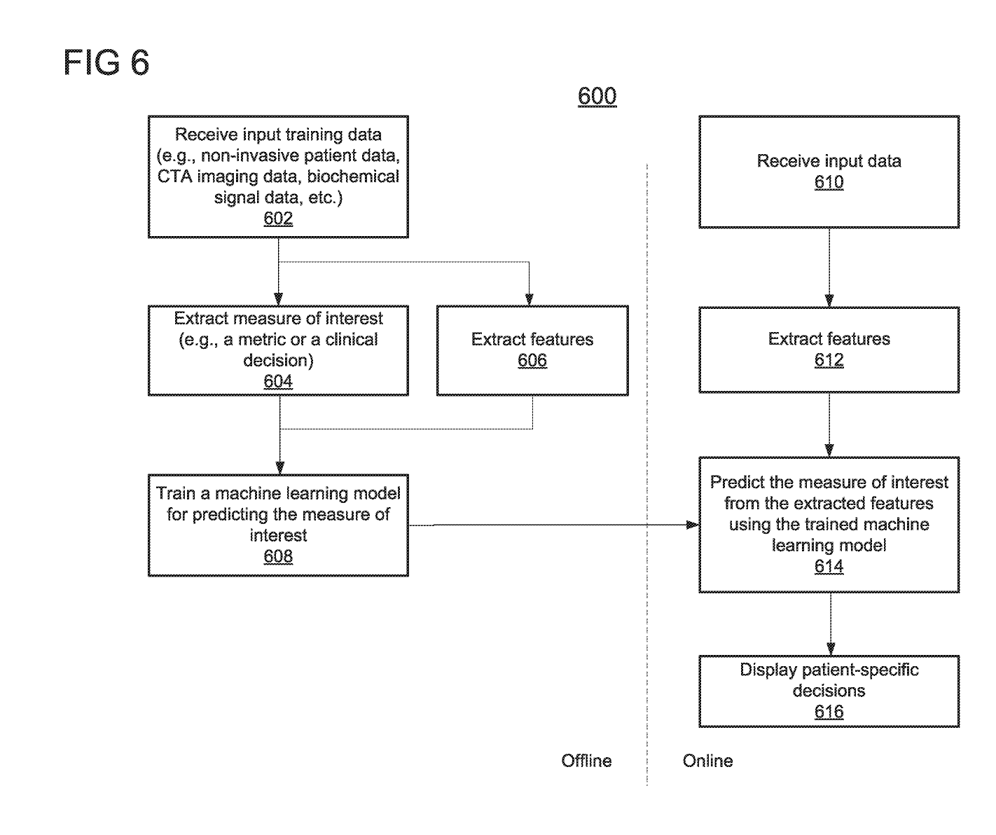

The first additional clinical test may include a CT-based test, such as, e.g., an artificial intelligence test, a rules based test, a computational modeling test, or any other suitable test. In one embodiment, the first additional clinical test is a test utilizing data of the CTA imaging. For example, the first additional clinical test may be one or more machine learning models trained to predict a measure of interest (e.g., a CT-FFR value, whether or not to perform CT-FFR, whether or not a patient has significant CAD, whether or not CT-FFR will be negative in all locations, etc.), such as the exemplary machine learning model trained and applied as described below with respect to FIGS. 6-8.

The first additional clinical test has a range of results 204. Similar to range of results 202, range of results 204 comprises a lower cutoff value delineating results 214 that provide the medical evaluation ruling out CAD and results 216 that do not provide the medical evaluation ruling out (or ruling in) CAD. An upper cutoff value of range of results 204 delineates results 218 that provide the medical evaluation ruling in CAD and results 216 that do not provide the medical evaluation ruling out (or ruling in) CAD. A result of the CTA performed on the patient within results 216 would not provide the medical evaluation ruling in or ruling out CAD and would therefore be a candidate for a second additional clinical test.

The second additional clinical test may be, e.g., a CT-fractional flow reserve (FFR) test. Similar to range of results 202, range of results 206 comprises a lower cutoff value delineating results 220 that provide the medical evaluation ruling out CAD and results 222 that do not provide the medical evaluation ruling out (or ruling in) CAD. An upper cutoff value of range of results 206 delineates results 224 that provide the medical evaluation ruling in CAD and results 222 that do not provide the medical evaluation ruling out (or ruling in) CAD. A result of the CTA within results 222 would not provide the medical evaluation ruling in or ruling out CAD and would therefore be a candidate for a third additional clinical test (e.g., invasive measurements of FFR). It should be understood that workflow 200 may proceed for any number of additional tests.

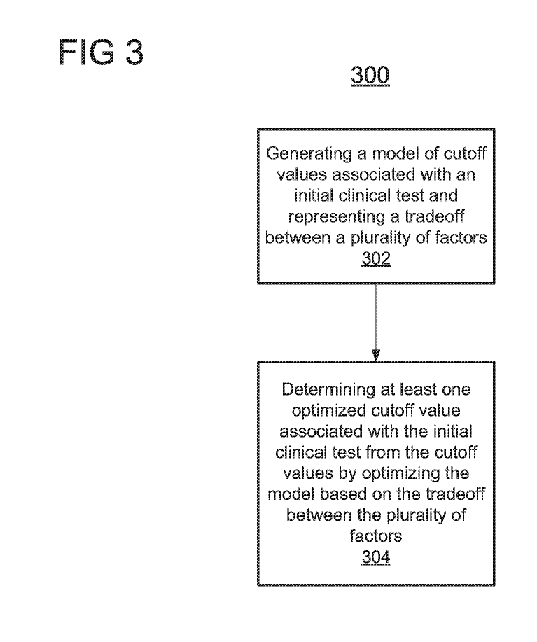

In one embodiment, optimized lower and upper cutoff values are determined for the lower and upper cutoff values for ranges of results 202, 204, and 206 are determined by separately performing the steps of method 300 of FIG. 3 for each clinical test. The optimized lower and upper cutoff values are determined as a tradeoff between a different factors, such as, e.g., a statistical measure (e.g., accuracy, sensitivity, specificity), patient outcome, and costs.

FIG. 3 shows a method 300 for determining optimized cutoff values for an initial clinical test, in accordance with one or more embodiments. The optimized cutoff values may provide clinical decision support for determining whether an additional clinical test should be performed on the patient. In one embodiment, optimization module 106, implemented on workstation 102 of FIG. 1, performs at least some of the steps of method 300 of FIG. 3. The steps of method 300 may be separately performed for each clinical test being performed on the patient in order to determine optimized cutoff values for that clinical test. For example, the steps of method 300 may be separately performed for each of the initial clinical test, the first additional clinical test, and the second additional clinical test discussed with respect to FIG. 2 to determine optimized lower and upper cutoff values for range of results 202, 204, and 206.

At step 302, a model of cutoff values associated with an initial clinical test and representing a tradeoff between a plurality of factors is generated from patient data. The patient data may include patient demographics, test results, medical equipment, clinical decisions and associated outcomes, or any other suitable type of patient data. The patient data may be historical patient data collected from a particular population of patients (e.g., to reflect the population characteristics in a particular hospital, region, country, etc.), clinical trials, screen processes, etc. In one embodiment, the patient data include information from CTA imaging, such as, e.g., calcium, plaque, degree and morphology of stenosis, etc. In one embodiment, the patient data is limited to data already available or which can be determined at no or very little cost, thereby providing for cost-effectiveness.

The initial clinical test is associated with a range of possible results. Lower and upper cutoff values define a boundary within the range of possible results of the initial clinical test delineating results that provide a medical evaluation with an acceptable level of uncertainty and results that do not provide the medical evaluation with an acceptable level of uncertainty. Each of the cutoff values represents a tradeoff between a plurality of factors, such as, e.g., a statistical measure of the initial clinical test (e.g., accuracy, sensitivity, or specificity), an overall cost for diagnosing and treating the patient, and a patient outcome of the initial clinical test. For example, an increase in the accuracy, sensitivity, or specificity of the initial clinical test will modify the cutoff values towards labeling less patients having a result that provides a medical evaluation, thereby increasing the overall cost for diagnosing and treating the patient (since more patients will be sent for additional clinical tests) but increasing patient outcome by reducing false negatives.

In one embodiment, the model of cutoff values associated with the initial clinical test is a three-dimensional surface model of the cutoff values. The surface model may be generated using any suitable approach. Exemplary methods for generating the surface model include, e.g., Delaunay based methods (which typically involving the computation of a Delaunay complex or of dual structures using scattered data and the extraction of the surface model from a previously computed Delaunay complex), volumetric methods (which approximate scattered data by a three dimensional function and extract the surface model as the zero-level set of the computed function), moving least squares (MLS) methods (which implicitly define the surfaces by the MLS projection operator), zippering meshes obtained from range images, statistical learning, polynomial chaos expansion, stochastic collocation, etc.

Noise may be superimposed during the generation of the model. Any suitable approach may be used to remove the noise. In addition, uncertainty may vary over different regions of the model. For example, uncertainty may be lower in regions with high point density and higher in regions with lower point density. Any suitable technique may be employed to account for uncertainty and to address issues associated with surface reconstruction from incomplete data.

At step 304, at least one optimized cutoff value is determined from the cutoff values by optimizing the model based on the tradeoff between the plurality of factors. In one embodiment, step 304 is repeated to find an optimized lower cutoff value and an optimized upper cutoff value for the initial clinical test.

In one embodiment, the model is optimized by optimizing a cost function f(statistical measure, cost, outcome), where the statistical measure refers to any statistical measure associated with the initial clinical test (e.g., accuracy, sensitivity, or specificity), the cost refers to the overall cost for treating the patient (including all clinical tests performed for diagnosing and treating the patient), and outcome refers to the outcome of the medical evaluation (e.g., positive or negative for CAD). In one embodiment, the cost may be based on the insurance of the patient. For example, the cost may be the out of pocket cost to the patient, without considering the cost paid by insurance. Each of the plurality of factors may be associated with a weight defining the tradeoff between the factors. The weights may be determined by the clinician, the patient, the payer of the cost, or any other entity.

In one embodiment, the function is:

f=w₁(100−accuracy)²+w₂(events)²+w₃(cost)²

where accuracy is the accuracy of the clinical test as a percentage, events is the rate of medical events, and cost is the cost per patient per day. In one embodiment, the medical events may include significant medical events, such as, e.g., death, myocardial infarction, hospitalization, invasive procedures, radiation exposure, or a major procedural complication. In another embodiment, the medical events are defined as MACE (major adverse cardiac events). Scaling factors w₁, w₂, and w₃ are scaling factors used to balance out differences in orders of magnitude and to weight the accuracy, events, and cost according to one or more criteria. An optimized cutoff value for the initial clinical test is determined by minimizing the function. Any suitable method may be used to optimize the function, such as, e.g., direct or iterative optimization, global or local optimization, etc. In some embodiments, the function may be a multi-objective function comprising, e.g., multiple cost functions.

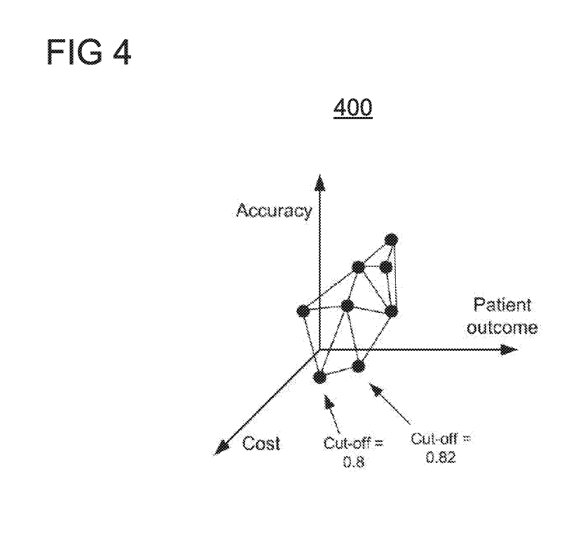

Referring for a moment to FIG. 4, an exemplary three dimensional surface model 400 is shown, in accordance with one or more embodiments. Surface model 400 is shown in three dimensional space with each axis representing one of the plurality of factors (e.g., accuracy, cost, and patient outcome in FIG. 4). Each point on surface model 400 corresponds to a different cutoff value. Surface model 400 provides a visual representation of the relationship between the plurality of factors associated with different cutoff values. As shown in FIG. 4, optimized cutoff values are determined as 0.80 and 0.83 representing the optimized lower and upper cutoff values, respectively.

It should be understood that while the embodiments discussed herein are described to balance three factors (e.g., accuracy, cost, and patient outcome), it should be understood that any number of factors may be considered. For example, the model of cutoff values may be a two dimensional model to balance two factors.

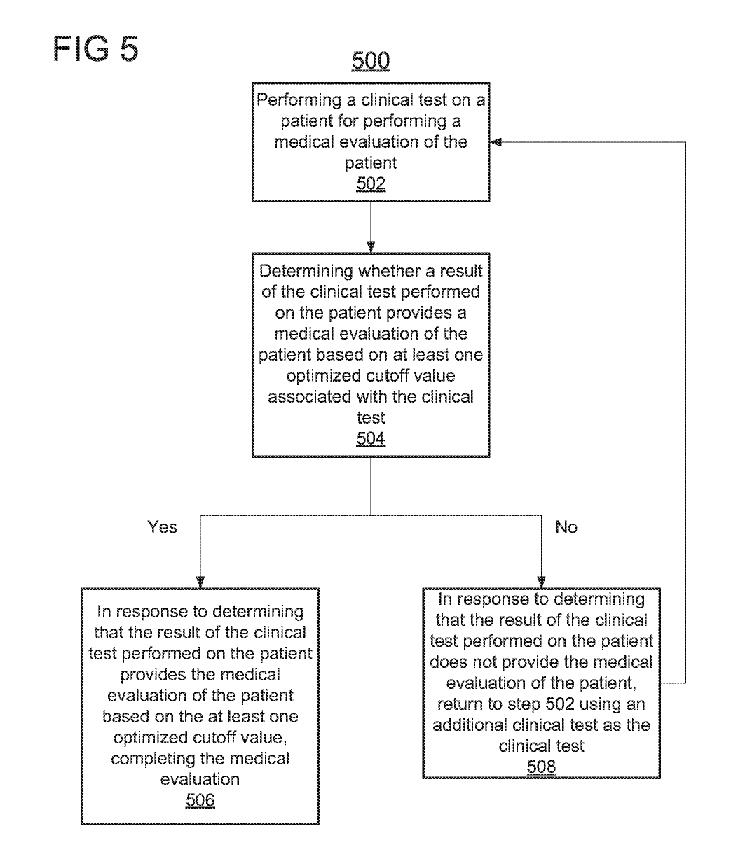

A determination is made whether to perform an additional test on the patient based on the result of the initial clinical test performed on the patient and the one or more cutoff values. For example, where the result of the initial clinical test is within the range of results that provide a medical evaluation (i.e., below an optimized lower cutoff value or above an optimized upper cutoff value), the medical evaluation of the patient can be completed and an additional clinical test is not performed. However, where the result of the initial clinical test is within the range of results that does not provide the medical evaluation (i.e., between the lower and the upper cutoff values), the additional clinical test may be performed. The upper and lower optimized cutoff values aid a clinician (or any other entity) in deciding whether an additional clinical test should be performed in order to perform the medical evaluation or whether the result of the clinical test provides the medical evaluation, in accordance with the tradeoffs between the plurality of factors. This is further discussed with respect to method 500 of FIG. 5.

Referring now to FIG. 5, a method 500 for performing a medical evaluation on a patient is shown, in accordance with one or more embodiments. The medical evaluation in method 500 is performed by applying one or more clinical tests.

At step 502, a clinical test is performed on the patient for performing a medical evaluation of the patient. For example, the clinical test may be CTA for the medical evaluation of diagnosis a patient with CAD, a CT-based test, the CT-FFR, etc.

At step 504, it is determined whether a result of the clinical test performed on the patient provides a medical evaluation of the patient based on at least one optimized cutoff value associated with the clinical test. In one embodiment, the at least one optimized cutoff value comprises an optimized lower cutoff value and an optimized upper cutoff value. In one embodiment, the at least one optimized cutoff value associated with the clinical test are determined by performing the steps of method 300 of FIG. 3 for the clinical test.

At step 506, in response to determining that the result of the clinical test performed on the patient provides the medical evaluation of the patient based on the at least one optimized cutoff value, the medical evaluation is completed and method 500 ends.

At step 508, in response to determining that the result of the clinical test performed on the patient does not provide the medical evaluation of the patient based on the at least one optimized cutoff value, method 500 returns to step 502 using an additional clinical test as the clinical test. In one embodiment, every iteration of method 500 resulting from step 510 provides for an additional clinical test of increasing expense (e.g., cost, time, complexity, etc.). For example, in one embodiment, the initial clinical test is CTA, a first additional clinical test is a CT-based test, a second additional clinical test is a machine learning model, and a fourth additional clinical test is invasive measurements of a patient to compute FFR. As such, the clinical tests are hierarchically applied in a manner to reduce unnecessary expense. In another embodiment, the clinical tests are limited by the patient data that is available or which can be determined at little or no cost, thereby providing for cost-analysis and cost-based decision making. Step 510 may be performed to return to step 502, e.g., until the result of the clinical test performed on the patient provides the medical evaluation of the patient, for a predetermined number of times, etc. Other stopping criteria may also be employed.

It should be understood that steps described herein may be performed according to the laws and rules of the applicable country, region, or other jurisdiction.

FIG. 6 shows a workflow 600 for training and applying a machine learning model for predicting a measure of interest, in accordance with one or more embodiments. In one embodiment, the trained machine learning (ML) model resulting from workflow 600 may be performed as the first additional clinical test in FIG. 2 or as the clinical test (or any additional clinical tests) at step 502 and/or step 504 in FIG. 5. Steps 602-608 show an offline or training stage for training a machine learning model and steps 610-616 show an online stage for applying the trained machine learning model to predict a measure of interest.

During an offline training stage, at step 602, input training data is received. The input training data may be any suitable data for training a machine learning model to predict a measure of interest. The measure of interest may be any suitable measure of interest to be predicted.

In one embodiment, the measure of interest is a result of a clinical test. For example, the measure of interest may be a predicted bin-based CT-FFR value (e.g., each bin spanning an interval of 0.1) or a prediction of whether the CT-FFR will be below a lower cutoff value, above an upper cutoff value, or between the lower and upper cutoff values (for predetermined, fixed cutoff values). In another example, the measure of interest may be a stenosis grade, risk of plaque rupture, or any other metric.

In another embodiment, the measure of interest is a clinical decision. The measure of interest may be represented as a binary value, such as, e.g., whether or not to send the patient to the cathlab (catheterization laboratory) for further invasive testing and/or intervention (e.g., diagnostic catheter, percutaneous transepatic cholangiography, coronary artery bypass graft, etc.), whether or not to perform CT-FFR, whether or not a patient has significant CAD, whether or not CT-FFR will be negative in all locations, etc. In one example, the machine learning model may predict whether or not CT-FFR should be performed for certain branches. As a result, the clinician may be able to focus on the identified branches, shortening the total runtime for the CT-FFR test.

The clinical decision may be a continuous variable (e.g., data of a future screening exam) or a series of hierarchical decisions. For example, the series of hierarchical decisions may be: 1) send the patient to the cathlab for further invasive testing and/or intervention; or 2) do not send the patient to the cathlab and: 2a) send the patient for an additional non-invasive test (e.g., perfusion imaging, single-photon emission computed tomography, stress, echocardiography, etc.), or 2b) discharge the patient and prescribe medication. Alternatively, in case of more than two options, multiple option choices may be performed (e.g., using a multi-class classifier). Each decision may be followed by finer-grade options associated with that decision. For example, following decision 1) send the patient to the cathlab, options for the type of examination that should be performed in the cathlab (e.g., x-ray angiography, optical coherence tomography, intravascular ultrasound, invasive FFR or instantaneous wave-free ration, etc.) may be provided. In another example, following decision 2b) discharge the patient and prescribe medication, options for which type of medication should be prescribed, when the patient should return for a follow up or screening exam, etc. may be provided.

The contents of the input training data is based on the measure of interest that the input training data will train the machine learning model to predict. The input training data may be received from a database populated with actual data of one or more patients, synthetic data (e.g., synthetic images or synthetic anatomical models) generated by simulating a medical imaging system, and/or data generated from in vitro experiments. The input training data may be acquired at a single point in time or at different points in time. In one embodiment, the input training data may be the patient data discussed above with respect to step 302 of FIG. 3. In one embodiment, the input training data is limited to data already available or which can be determined at little to no additional cost.

In one embodiment, the input training data includes non-invasive patient data. The non-invasive patient data may include, e.g. demographics information (e.g., age, ethnicity, gender, weight, height, race, and BMI); patient history (e.g., diabetes, hypertension, hypercholesterolemia, smoking history, family history of CAD, prior myocardial infarction, prior percutaneous coronary intervention, prior coronary artery bypass grafting, angina type (e.g., stable, worsening, silent ischemia, other angina category)); medical equipment and device measurements (e.g., stethoscope, bloom pressure meter, laboratory diagnostics, non-medical grade devices (e.g., wearables), blood pressure, heart rate, and ECG signals); type of patient (e.g., stable/acute); pre-test probability of CAD (e.g., based on simple clinical characteristics, including chest pain classification, age, and gender, clinical likelihood that spontaneous CAD is present); results of previously performed non-invasive tests (e.g., myocardial perfusion imaging, multigated acquisition scan, radionuclide stress test and nuclear stress test, exercise stress test, electrocardiogram, stress/rest echocardiography); and/or clinical history of the patient (e.g., recent exposure to radiation from other clinical tests).

In one embodiment, the input training data may include data determined from CTA imaging of the patient. The input training data may include results of an analysis of the CTA imaging performed by a clinician or a trained machine learning model. In one example, the analysis of the CTA imaging may be, e.g., an anatomical evaluation of the coronary arteries to determine a stenosis grading, stenosis length, stenosis location, plaque characteristics (e.g., composition, size, high risk plaque characteristics, degree of positive remodeling), etc. In another example, the analysis of the CTA imaging may be, e.g., a physiological or functional evaluation of the coronary arteries to determine, e.g., an FFR, CFR (coronary flow reserve), IFR (instantaneous wave-free ratio), WSS (wall shear stress), OSI (oscillatory shear index), or other metrics of the CTA imaging (e.g., image quality, calcium score, TAG (transluminal contrast attenuation gradient), risk scores (e.g., segment stenosis score, segment involvement score, Framingham risk score, etc.)).

In one embodiment, the input training data may include biochemical signals. The biochemical signals may be produced, e.g., by blood tests and molecular measurements (e.g., proteomics, transcriptomics, genomics, metabolomics, lipidomics, epigenomics, etc.) and/or features extracted based on radiogeneomics (e.g., imaging biomarkers that are linked with genomics of a pathology).

In one embodiment, the input training data may be generated by analyzing initial data of the input training data to extract additional features relevant for the measure of interest to be predicted. The analysis of the initial data may be based on, e.g., machine learning techniques or physics based techniques, such as techniques described in U.S. Pat. No. 9,349,178, entitled “Synthetic Data-Driven Hemodynamic Determination in Medical Imaging,” the disclosure of which is incorporated herein by reference in its entirety. The additional features may include, e.g., an FFR at all locations of the coronary arterial tree, a type of disease (e.g., diffuse disease or focal disease (e.g., by inspecting a virtual pull back curve from the terminal segments of the anatomical model to the ostium), a number of functionally significant lesions (serial lesions), prevalence of CAD (e.g., number of branches affected, whether the main branch or side braches are affected). In one embodiment, uncertainty quantifications for physiological measures of interest may calculated and used as part of the input training data, for example, as described in U.S. Pat. No. 9,349,178.

In one embodiment, FFR computed from angiographic data is used as the ground truth values during the training. In another embodiment, if the predicted decision is not based on functional indices (e.g., measured or computed) and anatomical markers are used for decision making, the anatomical markers may be used as ground truth values.

At step 604, the measures of interest are extracted from the input training data. At step 606, features are extracted from the input training data. The features are determined based on the characteristics available (e.g., in the database) for the input training data. For example, the features may include genetic, radiogenomic, or other phenotype based features. In another example, the features may include non-invasive patient data, such as, e.g., demographics information, patient history, medical equipment and device measurements, type of patient, etc. It should be understood that step 606 may be performed at any time prior to step 608 (e.g., before step 604, after step 604, or concurrently with (e.g., in parallel with) step 604).

At step 608, one or more machine learning models are trained to predict the measures of interest. The machine learning models may be trained using any suitable approach, such as, e.g., regression, instance-based methods, regularization methods, decision tree learning, Bayesian, kernel methods, clustering methods, association rule learning, artificial neural networks, dimensionality reduction, ensemble methods, etc. In one embodiment, the machine learning models are trained using deep learning. Deep learning generally refers to a machine learning technique using several information processing layers. Hierarchical structures are employed, either for learning features or representations, or for classification or regression.

In one embodiment, the trained machine learning model may also include a confidence score along with potential sources of uncertainty. The model may suggest additional measurements/clinical tests which can increase the information content.

During the online stage, at step 610, input data is received. The input data received at this step represents unseen data of the patient to be imaged. The input data may be any suitable data for predicting the measures of interest by a trained machine learning model. For example, input data at step 610 may include non-invasive patient data, CTA based data, biochemical signals, or other data such as described above with respect to step 602.

At step 612, features are extracted from the input data. At block 614, measures of interest are predicted from the (i.e., based on the) extracted features using the trained machine learning model. The measures of interest predict whether CT-FFR is required.

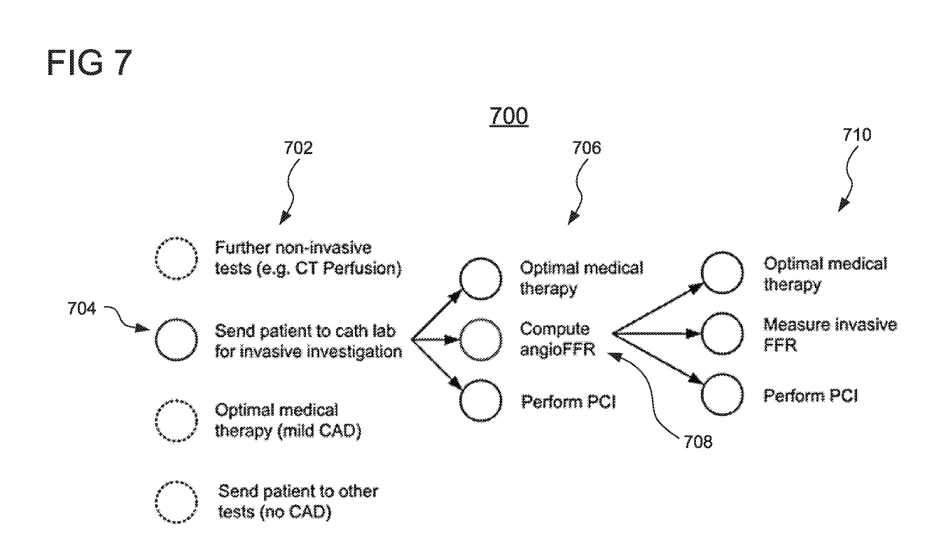

At step 616, patient-specific decisions are displayed using a display device. In one embodiment, a text based display is used to display the patient-specific decisions. For example, the patient-specific decisions may include the characteristics or features that were most important for the prediction (e.g., calcium score>400, age>65→CT-FFR test not required since the probability for cathlab intervention is higher than 95%). In another embodiment, the currently the selected clinical decision and possible subsequent clinical decisions are displayed as a hierarchy. Referring for a moment to FIG. 7, a user interface 700 is shown in accordance with one or more embodiments. User interface 700 shows available options 702, from which option 704 is selected. Subsequent options 706 are shown for selected option 704. Similarly, a user may select option 708 from the subsequent options 706 and subsequent options 710 of selected option 708 are displayed. A user may interact with user interface 700 (via a input device, such as, e.g., a mouse, a keyboard, touchscreen device, etc.) to select, rotate, zoom, pan, etc. User interface 700 allows a user to view an evolution of a patient over time.

In one embodiment, where the predicted measure of interest in FIG. 6 is predicted CT-FFR values, step 502 of FIG. 5 is performed by performing the steps of the online stage (i.e., steps 610-616). Accordingly, the predicted CT-FFR values may be used in step 504 of FIG. 5 to determine whether the predicted CT-FFR values provides a medical evaluation of the patient based on at least one optimized cutoff value. In another embodiment, where the predicted measure of interest in FIG. 6 is a binary value (e.g., whether or not to perform CT-FFR, whether or not a patient has significant CAD, whether or not CT-FFR will be negative in all locations), steps 502 and 504 of FIG. 5 may be performed by performing the steps of the online stage (i.e., steps 610-616).

In one embodiment, the trained machine learning model is trained for semi-automated decision making. For example, the clinician may be presented with, and may select from, a number of possible decisions and/or subsets of those decisions. The clinician may select the decisions based on his or her experience and judgment or present circumstances (e.g., a certain type of invasive test may not be available at the hospital). Multiple machine learning models may be trained for different subsets of possible decisions, or the same machine learning model may be employed irrespective of the elected decision, and the possible decision with the highest probably from the selected set may be suggested. In one embodiment, the clinician may intervene in the workflow by choosing to discard some of the received input data (at step 610) that he or she considers less relevant. In one embodiment, instead of outputting a specific decision, the trained machine learning model may be used to present to the clinician a top n (e.g., top 3) possible decisions, ranked based on their corresponding confidence. The clinician can select the final decision.

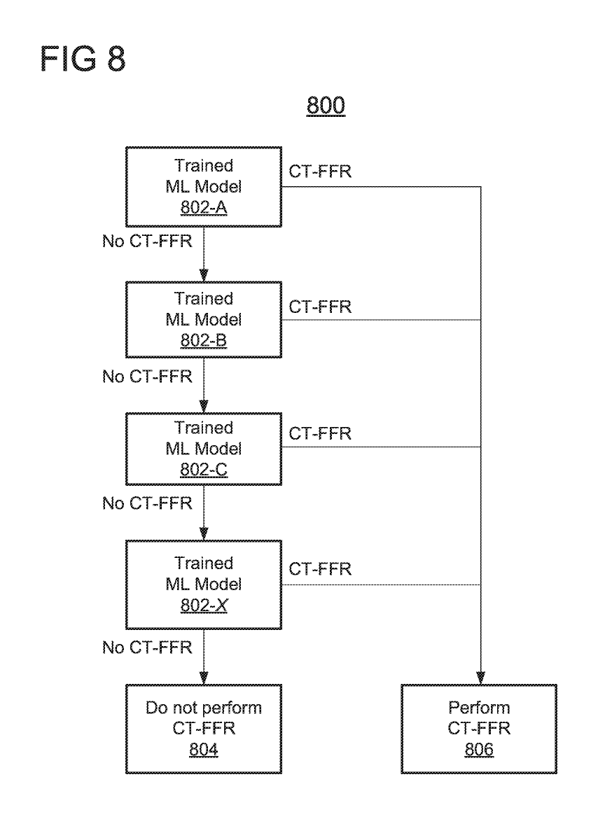

In one embodiment, a plurality of machine learning models may be hierarchically applied, each of increasing expense. Referring now to FIG. 8, a workflow 800 is shown for hierarchically applying a cascade of trained machine learning models, in accordance with one or more embodiments. In one embodiment, the workflow 800 may be performed as the first additional clinical test in FIG. 2 or as the clinical test (or any additional clinical tests) at step 502 and step 504 in FIG. 5.

Workflow 800 comprises a cascade of trained machine learning models 802-A, 802-B, 802-C, . . . , and 802-X (collectively referred to herein as machine learning models 702). While workflow 800 is shown having four machine learning models 802, it should be understood that machine learning models 802 may comprise any number of machine learning models. In one embodiment, some or all of machine learning models 802 may be separately trained in an offline stage by performing steps 602-608 in FIG. 6 and may be applied in an online stage by performing steps 610-616 in FIG. 6.

Each machine learning model 802 is trained to predict a measure of interest, such as, e.g., whether or not to perform CT-FFR, using different input training data. In one embodiment, each machine learning model 802 is trained with progressively more input training data than a previous machine learning model 802. For example, each machine learning model 802 may be trained with progressively more expensive (e.g., computationally, monetarily, time, invasiveness, etc.) training input data.

In one embodiment, machine learning model 802-A is trained using demographic information and patient history information; machine learning model 802-B is trained using the training input data used in training machine learning model 802-A in addition to non-invasive test information; machine learning model 802-C is trained using the training input data used in training machine learning model 802-B in addition to calcium scoring test information; and machine learning model 802-X is trained using the training input data used in training machine learning model 802-C in addition to CTA-based data (e.g., anatomical information from CTA imaging). Accordingly, during an online stage, machine learning models 802-A and 802-B may be executed prior to acquiring CTA imaging data, machine learning model 802-C may be executed after a calcium scoring test, and machine learning model 802-X may be executed after performing an anatomical evaluation of the coronary arteries. Each machine learning model 802 in workflow 800 is progressively more expensive to execute (in terms of e.g., computation, cost, time, invasiveness, etc.).

During the online stage, workflow 800 successively applies each machine learning model 802 of the cascade until, e.g., it is determined to perform CT-FFR. In this manner, machine learning model 802-A is first executed. If machine learning model 802-A predicts that CT-FFR should not be performed, workflow 800 proceeds to execute a next machine learning model 802-B. Each remaining model of machine learning model 802 executes in a similar manner, such that if a machine learning model 802 predicts that CT-FFR should not be performed, workflow 800 proceeds to execute a next model of machine learning model 702. If a last model of machine learning model 802 (i.e., machine learning model 802-X) predicts that CT-FFR should not be performed, workflow 800 proceeds to step 804 where CT-FFR is not performed and workflow 800 ends. However, if any machine learning model 802 predicts that CT-FFR should be performed, workflow 800 proceeds to step 806 to perform CT-FFR and workflow 800 ends.

While workflow 800 is described for predicting whether to run CT-FFR, it should be understood that workflow 800 may be employed to predict any suitable measure of interest, such as, e.g., whether or not a patient has significant CAD or whether or not CT-FFR will be negative in all locations. In one embodiment, workflow 800 may be modified for machine learning models trained to predict CT-FFR values and associated measures of uncertainty (or level of confidence). Accordingly, in this embodiment, if a machine learning model predicts a CT-FFR value with an uncertainty that does not satisfy a threshold (e.g., below a threshold), the workflow proceeds to a next machine learning model. If a last machine learning model in the workflow predicts a CT-FFR value with an uncertainty that does not satisfy the threshold, CT-FFR is performed. However, if any of the machine learning models predicts a CT-FFR value that satisfies the threshold, workflow 800 ends and the predicted CT-FFR value is output. For example, the predicted CT-FFR value may be used at step 504 of FIG. 5 to determine if an additional clinical test should be performed.

In one embodiment of the embodiments described herein, a machine learning model may be trained to predict a measure of interest to determine whether to perform CT-FFR using partial information (gathered while performing CT-FFR) as the input training data. For example, the measure of interest may be whether CAD can be excluded, whether CAD is functionally significant (in which case the patient would be sent to the cath lab), etc. The machine learning model in this embodiment may be trained by performing steps 602-608 of FIG. 6. In one embodiment, input training data may partial information, such as, e.g., anatomical information extracted using fully automated centerline and segmentation algorithms, anatomical information of main branches, a subset of features that are used by the CT-FFR machine learning based model (e.g., a set of features that can be determined automatically or with limited user interaction), etc. Accordingly, during the online stage, machine learning models may aid a decision to stop performing the CT-FFR test during the test (e.g., because CAD can be excluded or because CAD is functionally significant). This embodiment is particularly advantageous when the CT-FFR test cannot be excluded a priori.

In one embodiment, the decision of whether to perform CT-FFR is based on a measure of “utility” of CT-FFR. The utility may reflect, for example, the expected amount of time before the CT-FFR results are available as compared to the state of the patient. For example, in an emergency situation (e.g., in the emergency room) or for an unstable patient, the additional wait time for performing CT-FFR may increase the risk for the patient (e.g., prolonged ischemia), in which case alternative tests (invasive FFR) may be recommended regardless of the expected CT-FFR result.

In one embodiment, the embodiments described herein may be applied for both stable and acute CAD patients. In one embodiment, optimization module 116 in FIG. 1 may be executed on workstation 102 onsite (e.g., at a hospital or clinic) but may additionally or alternatively be executed offsite, e.g., for additional processing power. In one embodiment, machine learning models executed on a workstation onsite may be used as a gatekeeper for offsite execution of machine learning models. In one embodiment, the embodiments described herein may be applied for optimizing the scheduling of patients for CT exams (the CT-FFR test may only be available at certain times). In one embodiment, instead of machine learning models, any other suitable decision making structure may be employed (e.g., rule based model or reduced-order model). In one embodiment, the embodiments described herein may be applied to dual energy and photon counting based imaging.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps of the methods and workflows described herein, including one or more of the steps of FIGS. 2, 4, and 8-13. Certain steps of the methods and workflows described herein, including one or more of the steps of FIGS. 2, 4, and 8-13, may be performed by a server or by another processor in a network-based cloud-computing system. Certain steps of the methods and workflows described herein, including one or more of the steps of FIGS. 3, 5, 6, and 8, may be performed by a client computer in a network-based cloud computing system. The steps of the methods and workflows described herein, including one or more of the steps of FIGS. 3, 5, 6, and 8, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps of FIGS. 3, 5, 6, and 8, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 902 that may be used to implement systems, apparatus, and methods described herein is depicted in FIG. 9. Computer 902 includes a processor 904 operatively coupled to a data storage device 912 and a memory 910. Processor 904 controls the overall operation of computer 902 by executing computer program instructions that define such operations. The computer program instructions may be stored in data storage device 912, or other computer readable medium, and loaded into memory 910 when execution of the computer program instructions is desired. Thus, the method and workflow steps of FIGS. 3, 5, 6, and 8 can be defined by the computer program instructions stored in memory 910 and/or data storage device 912 and controlled by processor 904 executing the computer program instructions. For example, the computer program instructions can be implemented as computer executable code programmed by one skilled in the art to perform the method and workflow steps of FIGS. 3, 5, 6, and 8. Accordingly, by executing the computer program instructions, the processor 904 executes the method and workflow steps of FIGS. 3, 5, 6, and 8. Computer 904 may also include one or more network interfaces 906 for communicating with other devices via a network. Computer 902 may also include one or more input/output devices 1408 that enable user interaction with computer 902 (e.g., display, keyboard, mouse, speakers, buttons, etc.).

Processor 904 may include both general and special purpose microprocessors, and may be the sole processor or one of multiple processors of computer 902. Processor 904 may include one or more central processing units (CPUs), for example. Processor 904, data storage device 912, and/or memory 910 may include, be supplemented by, or incorporated in, one or more application-specific integrated circuits (ASICs) and/or one or more field programmable gate arrays (FPGAs).

Data storage device 912 and memory 910 each include a tangible non-transitory computer readable storage medium. Data storage device 912, and memory 910, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 908 may include peripherals, such as a printer, scanner, display screen, etc. For example, input/output devices 908 may include a display device such as a cathode ray tube (CRT) or liquid crystal display (LCD) monitor for displaying information to the user, a keyboard, and a pointing device such as a mouse or a trackball by which the user can provide input to computer 902.

Any or all of the systems and apparatus discussed herein, including elements of workstation 102 of FIG. 1, may be implemented using one or more computers such as computer 902.

One skilled in the art will recognize that an implementation of an actual computer or computer system may have other structures and may contain other components as well, and that FIG. 9 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method, comprising:
generating a model of cutoff values associated with an initial clinical test and representing a tradeoff between a plurality of factors, each of the cutoff values defining a boundary within a range of results of the initial clinical test delineating results that provide a medical evaluation and results that do not provide the medical evaluation;
determining at least one optimized cutoff value associated with the initial clinical test from the cutoff values by optimizing the model based on the tradeoff between the plurality of factors; and
determining whether to perform an additional clinical test based on a result of the initial clinical test performed on the patient and the at least one optimized cutoff value.

2. The method of claim 1, wherein the plurality of factors comprises accuracy of the initial clinical test, cost of the initial clinical test, and patient outcome of the initial clinical test.

3. The method of claim 1, wherein determining whether to perform an additional clinical test based on a result of the initial clinical test performed on the patient and the at least one optimized cutoff value comprises:
a) performing the initial clinical test on a patient;
b) determining whether a result of the initial clinical test performed on the patient provides a medical evaluation of the patient based on the at least one optimized cutoff value; and
c) in response to determining that the result of the initial clinical test performed on the patient does not provide the medical evaluation of the patient, repeating steps a) and b) using a respective additional clinical test as the initial clinical test until it is determined that the result of the respective clinical test performed on the patient provides the medical evaluation of the patient or for a predetermined number of iterations.

4. The method of claim 3, wherein the at least one optimized cutoff value associated with the initial clinical test comprises:
an optimized lower cutoff value delineating results below the optimized lower cutoff value that provide the medical evaluation and results above the optimized lower cutoff value that do not provide the medical evaluation; and
an optimized upper cutoff value delineating results above the optimized upper cutoff value that provide the medical evaluation and results below the optimized upper cutoff value that do not provide the medical evaluation.

5. The method of claim 4, wherein determining whether a result of the initial clinical test performed on the patient provides a medical evaluation of the patient based on the at least one optimized cutoff value comprises:
determining that the result of the initial clinical test performed on the patient does not provide the medical evaluation of the patient when the result of the initial clinical test is above the optimized lower cutoff value and below the optimized upper cutoff value.

6. The method of claim 1, wherein the additional clinical test is more expensive to perform on the patient than the initial clinical test.

7. The method of claim 1, wherein the initial clinical test comprises at least one trained machine learning model.

8. The method of claim 7, wherein the at least one trained machine learning model is trained to predict the result of the initial clinical test or whether to apply the additional clinical test.

9. The method of claim 7, wherein the at least one trained machine learning model comprises a cascade of trained machine learning models, wherein the trained machine learning models in the cascade are successively applied until it is determined whether to apply the additional clinical test.

10. An apparatus, comprising:
means for generating a model of cutoff values associated with an initial clinical test and representing a tradeoff between a plurality of factors, each of the cutoff values defining a boundary within a range of results of the initial clinical test delineating results that provide a medical evaluation and results that do not provide the medical evaluation;
means for determining at least one optimized cutoff value associated with the initial clinical test from the cutoff values by optimizing the model based on the tradeoff between the plurality of factors; and
means for determining whether to perform an additional clinical test based on a result of the initial clinical test performed on the patient and the at least one optimized cutoff value.

11. The apparatus of claim 10, wherein the plurality of factors comprises accuracy of the initial clinical test, cost of the initial clinical test, and patient outcome of the initial clinical test.

12. The apparatus of claim 10, wherein the means for determining whether to perform an additional clinical test based on a result of the initial clinical test performed on the patient and the at least one optimized cutoff value comprises:
a) means for performing the initial clinical test on a patient;
b) means for determining whether a result of the initial clinical test performed on the patient provides a medical evaluation of the patient based on the at least one optimized cutoff value; and
c) in response to determining that the result of the initial clinical test performed on the patient does not provide the medical evaluation of the patient, means for repeating steps a) and b) using a respective additional clinical test as the initial clinical test until it is determined that the result of the respective clinical test performed on the patient provides the medical evaluation of the patient or for a predetermined number of iterations.

13. The apparatus of claim 12, wherein the at least one optimized cutoff value associated with the initial clinical test comprises:
an optimized lower cutoff value delineating results below the optimized lower cutoff value that provide the medical evaluation and results above the optimized lower cutoff value that do not provide the medical evaluation; and
an optimized upper cutoff value delineating results above the optimized upper cutoff value that provide the medical evaluation and results below the optimized upper cutoff value that do not provide the medical evaluation.

14. The apparatus of claim 13, wherein the means for determining whether a result of the initial clinical test performed on the patient provides a medical evaluation of the patient based on the at least one optimized cutoff value comprises:
means for determining that the result of the initial clinical test performed on the patient does not provide the medical evaluation of the patient when the result of the initial clinical test is above the optimized lower cutoff value and below the optimized upper cutoff value.

15. A non-transitory computer readable medium storing computer program instructions, the computer program instructions when executed by a processor cause the processor to perform operations comprising:
generating a model of cutoff values associated with an initial clinical test and representing a tradeoff between a plurality of factors, each of the cutoff values defining a boundary within a range of results of the initial clinical test delineating results that provide a medical evaluation and results that do not provide the medical evaluation;
determining at least one optimized cutoff value associated with the initial clinical test from the cutoff values by optimizing the model based on the tradeoff between the plurality of factors; and
determining whether to perform an additional clinical test based on a result of the initial clinical test performed on the patient and the at least one optimized cutoff value.

16. The non-transitory computer readable medium of claim 15, wherein the operation of determining whether to perform an additional clinical test based on a result of the initial clinical test performed on the patient and the at least one optimized cutoff value comprises:
a) performing the initial clinical test on a patient;
b) determining whether a result of the initial clinical test performed on the patient provides a medical evaluation of the patient based on the at least one optimized cutoff value;
c) in response to determining that the result of the initial clinical test performed on the patient does not provide the medical evaluation of the patient, repeating steps a) and b) using a respective additional clinical test as the initial clinical test until it is determined that the result of the respective clinical test performed on the patient provides the medical evaluation of the patient or for a predetermined number of iterations.

17. The non-transitory computer readable medium of claim 15, wherein the additional clinical test is more expensive to perform on the patient than the initial clinical test.

18. The non-transitory computer readable medium of claim 15, wherein the initial clinical test comprises at least one trained machine learning model.

19. The non-transitory computer readable medium of claim 18, wherein the at least one trained machine learning model is trained to predict the result of the initial clinical test or whether to apply the additional clinical test.

20. The non-transitory computer readable medium of claim 18, wherein the at least one trained machine learning model comprises a cascade of trained machine learning models, wherein the trained machine learning models in the cascade are successively applied until it is determined whether to apply the additional clinical test.
